(19) United States
(12) Patent Application Publication
(10) Pub. No.: US 2025/0221784 A1
(43) Pub. Date: Jul. 10, 2025

(54) GRIP FORCE CONTROL IN A SURGICAL ROBOTIC SYSTEM

(71) Applicant: CMR Surgical Limited, Cambridge (GB)

(72) Inventor: David William Haydn Webster-Smith, Cambridge (GB)

(73) Assignee: CMR Surgical Limited, Cambridge, OT (GB)

(21) Appl. No.: 18/847,132

(22) PCT Filed: Mar. 20, 2023
(86) PCT No.: PCT/GB2023/050666

§ 371 Date: Sep. 13, 2024

(30) Foreign Application Priority Data
Mar. 21, 2022 (GB) 2203946.5

(51) Int. Cl. A61B 90/00; A61B 17/29; A61B 34/30

(52) U.S. Cl. CPC A61B 90/03; A61B 34/30; A61B 17/29

(57) ABSTRACT

A grip force controller controls grip force of an instrument in a surgical robotic system. The surgical robotic system includes a robot with a base and an arm extending from the base to a drive assembly. The instrument has an end effector including a plurality of jaws. The drive assembly transfers drive to the plurality of jaws. The grip force controller is configured to control movement of the jaws according to a demanded spread between the jaws; determine an occurrence of a gripping event as the jaws are driven relative to each other in the closing direction so as to apply a grip force between the jaws; determine a demanded spread between the jaws on determining the occurrence of the gripping event; set the determined demanded spread as a minimum spread limit for the jaws; and control movement of the jaws using the minimum spread limit.

### FIELD OF THE INVENTION

This invention relates to controlling the grip of a jawed instrument of a surgical robotic system for achieving a more consistent grip force of the jaws.

### BACKGROUND

It is known to use robots for assisting and performing surgery. FIG. 1 illustrates a typical surgical robot 100 which consists of a base 108 and an arm 102. An instrument 105 is coupled to the arm.

The base supports the robot, and is itself attached rigidly to, for example, the operating theatre floor, the operating theatre ceiling or a trolley. The arm extends between the base and the instrument. The arm is articulated by means of multiple flexible joints 103 along its length, which are used to locate the surgical instrument in a desired location relative to the patient. The surgical instrument is attached to the distal end 104 of the robot arm. The surgical instrument penetrates the body of the patient 101 at a port 107 so as to access the surgical site. At its distal end, the instrument comprises an end effector 106 for engaging in a medical procedure.

FIG. 2 illustrates a typical surgical instrument 200 for performing robotic laparoscopic surgery. The surgical instrument comprises an instrument interface 201 by means of which the surgical instrument connects to the robot arm. A shaft 202 extends between the interface 201 and an articulation 203. The articulation 203 terminates in an end effector 204. In FIG. 2, a pair of serrated jaws are illustrated as the end effector 204. The articulation 203 permits the end effector 204 to move relative to the shaft 202. It is desirable for at least two degrees of freedom to be provided to the motion of the end effector 204 by means of the articulation. The articulation permits the end effector to move in a yaw direction 205. The instrument jaws are movable, together and/or separately, in the yaw direction 205. The instrument interface is configured to couple to an arm drive assembly of the robot arm to which the instrument is engageable. When the instrument is engaged with the arm, the instrument interface engages with the drive assembly thereby to transfer drive to the instrument.

FIG. 3 illustrates a surgical robot having an arm 300 which extends from a base 301. The arm comprises a number of rigid limbs 302. The limbs are coupled by revolute joints 303. The most proximal limb 302a is coupled to the base by a proximal joint 303a. It and the other limbs are coupled in series by further ones of the joints 303. Suitably, a wrist 304 is made up of four individual revolute joints. The wrist 304 couples one limb (302b) to the most distal limb (302c) of the arm. The most distal limb 302c carries an attachment 305 for a surgical instrument 306. Each joint 303 of the arm has one or more motors 307 which can be operated to cause rotational motion at the respective joint, and one or more position and/or torque sensors 308 which provide information regarding the current configuration and/or load at that joint. Suitably, the motors are arranged proximally of the joints whose motion they drive, so as to improve weight distribution. For clarity, only some of the motors and sensors are shown in FIG. 3. The arm may be generally as described in patent application PCT/GB2014/053523 (WO 2015/132549).

The arm terminates in the attachment 305 for interfacing with the instrument 306. Suitably, the instrument 306 takes the form described with respect to FIG. 2. The instrument has a diameter less than 8 mm. Suitably, the instrument has a 5 mm diameter. The instrument may have a diameter which is less than 5 mm. The instrument diameter may be the diameter of the shaft. The instrument diameter may be the diameter of the profile of the articulation. Suitably, the diameter of the profile of the articulation matches or is narrower than the diameter of the shaft. The attachment 305 comprises a drive assembly for driving articulation of the instrument. Movable interface elements of the drive assembly mechanically engage corresponding movable interface elements of the instrument interface in order to transfer drive from the robot arm to the instrument. One instrument is exchanged for another several times during a typical operation. Thus, the instrument is attachable to and detachable from the robot arm during the operation. Features of the drive assembly interface and the instrument interface aid their alignment when brought into engagement with each other, so as to reduce the accuracy with which they need to be aligned by the user.

The instrument 306 comprises an end effector for performing an operation. The end effector may take any suitable form. For example, the end effector may be smooth jaws, serrated jaws, a gripper, a pair of shears, a needle for suturing, a camera, a laser, a knife, a stapler, a cauteriser, a suctioner. As described with respect to FIG. 2, the instrument comprises an articulation between the instrument shaft and the end effector. The articulation comprises several joints which permit the end effector to move relative to the shaft of the instrument. The joints in the articulation are actuated by driving elements, such as cables. These driving elements are secured at the other end of the instrument shaft to the interface elements of the instrument interface. Thus, the robot arm transfers drive to the end effector as follows: movement of a drive assembly interface element moves an instrument interface element which moves a driving element which moves a joint of the articulation which moves the end effector.

Controllers for the motors, torque sensors and encoders are distributed within the robot arm. The controllers are connected via a communication bus to a control unit 309. The control unit 309 comprises a processor 310 and a memory 311. The memory 311 stores in a non-transient way software that is executable by the processor to control the operation of the motors 307 to cause the arm 300 to operate in the manner described herein. In particular, the software can control the processor 310 to cause the motors (for example via distributed controllers) to drive in dependence on inputs from the sensors 308 and from a surgeon command interface 312. The control unit 309 is coupled to the motors 307 for driving them in accordance with outputs generated by execution of the software. The control unit 309 is coupled to the sensors 308 for receiving sensed input from the sensors, and to the command interface 312 for receiving input from it. The respective couplings may, for example, each be electrical or optical cables, and/or may be provided by a wireless connection. The command interface 312 comprises one or more input devices whereby a user can request motion of the end effector in a desired way. The input devices could, for example, be manually operable mechanical input devices such as control handles or joysticks, or contactless input devices such as optical gesture sensors. The software stored in the memory 311 is configured to respond to those inputs and cause the joints of the arm and instrument to move accordingly, in compliance with a pre-determined control strategy. The control strategy may include safety features which moderate the motion of the arm and instrument in response to command inputs. Thus, in summary, a surgeon at the command interface 312 can control the instrument 306 to move in such a way as to perform a desired surgical procedure. The control unit 309 and/or the command interface 312 may be remote from the arm 300.

The illustrated surgical robot comprises a single robot arm. Other surgical robot systems may comprise a plurality of surgical robots and/or a plurality of robot arms. For example, other example surgical robot systems may comprise a surgical robot with a plurality of robot arms that can each receive and manipulate a surgical instrument, or they may comprise a plurality of surgical robots that each have a robot arm that can receive and manipulate a surgical instrument.

The arm 102 terminates in an attachment, an example of which can be seen in FIG. 4, for interfacing with the instrument. The attachment comprises a drive assembly for driving articulation of the instrument 105. The drive assembly interface 400 interfaces with the instrument interface, an example of which is shown in FIG. 5 at 500. Moveable interface elements 401, 402, 403 of the drive assembly engage corresponding moveable interface elements 502 of the instrument interface in order to transfer drive from the robot arm 102 to the instrument 105. In the example shown in FIGS. 4 and 5, the drive assembly interface elements comprise protruding fins 401, 402, 403 and the interface elements of the instrument interface comprise cups 502 for receiving the fins. The fins and cups can be provided either way round. In some implementations, the drive assembly interface elements comprise cups and the interface elements of the instrument interface comprise fins receivable in the cups. Other ways in which respective drive assembly interface elements and respective instrument interface elements engage with one another may be provided. Linear motion of the drive assembly interface elements causes corresponding linear motion of the interface elements of the instrument interface.

The surgical robot 100 is controlled remotely by an operator (e.g. surgeon) via an operator console 600 shown in FIG. 6. The operator console 600 may be located in the same room (e.g. operating theatre) as the surgical robot 100 or remotely from it. The operator console 600 comprises input devices 602, 604 for controlling the state of the arm 102 and/or the instrument 105 attached thereto. The input devices 602, 604 may be handgrips or hand controllers mounted on parallelogram linkages. A control system converts the movement of the hand controllers into control signals to move the arms, joints and/or instrument end effector of a surgical robot. The operator console 600 also comprises a display 606. The display 606 is arranged to be visible to a user operating the input devices 602, 604. The display is used to display a video stream of the surgical site (e.g. endoscope video).

In some robotic control systems, including surgical robotic control systems, there can be variability in the grip force applied when a user controls a jawed end effector to close. The grip force may be applied when the jaws contact each other, or when they contact an object located between the jaws. An example of a jawed instrument is a needle holder. The needle holder will apply a grip force when a needle is gripped between the jaws. The grip can differ in dependence on the position along the jaws that the needle is gripped and on the thickness of the needle itself.

A user can give a command to close the jaws by operating a user input device. In response, the control system can output a demanded closure force between the jaws, for example, or specify a desired closure state of the jaws, e.g. a spread of the jaws. The spread can be defined by an angular separation of the jaws, or in any other convenient way. For a given demanded closure force and/or a given desired closure state of the jaws, the force that is actually applied can depend on the particular instrument used and/or the particular drive assembly used to drive that instrument. For example, manufacturing tolerances mean that each instrument and drive assembly may be slightly different, which can lead to the force variations. It is desirable to reduce the variation in force applied where the commanded state remains consistent.

### SUMMARY

This summary is provided to introduce a selection of concepts that are further described below in the detailed description. This summary is not intended to identify key features or essential features of the claimed subject matter, nor is it intended to be used to limit the scope of the claimed subject matter.

According to an aspect of the present invention, there is provided a grip force controller for controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the grip force controller being configured to:
control movement of the jaws according to a demanded spread between the jaws;
determine an occurrence of a gripping event as the jaws are driven relative to each other in the closing direction so as to apply a grip force between the jaws;
determine a demanded spread between the jaws on determining the occurrence of the gripping event;
set the determined demanded spread as a minimum spread limit for the jaws; and
control movement of the jaws using the minimum spread limit so as to control grip force applied by the jaws.

The grip force controller may be configured to determine the occurrence of the gripping event on determining that any one or more of the following occur: a grip force between the jaws exceeds a grip force threshold; a demanded motor force of a motor of the drive assembly for transferring drive to one of the jaws exceeds a motor force threshold; an average demanded motor force of a plurality of motors of the drive assembly for transferring drive to respective ones of the jaws exceeds an average motor force threshold; the motor of the drive assembly for transferring drive to one of the jaws approaches or reaches a saturation point; a current through the motor of the drive assembly for transferring drive to one of the jaws exceeds a current threshold; a driving element of the instrument, by which drive is transferred to one of the jaws, approaches or reaches a physical limit of the driving element; and a physical limit is reached of a component of the instrument.

The grip force controller may be configured to determine the occurrence of the gripping event based on a signal indicative of one or more of: the grip force between the jaws; the demanded motor force of the motor; the average demanded motor force of the plurality of motors; motor saturation of the motor of the drive assembly for transferring drive to one of the jaws; current through the motor of the drive assembly for transferring drive to one of the jaws; and tension in the driving element of the instrument by which drive is transferred to one of the jaws. The signal may comprise or may be derived from visual servoing based on an image of the instrument. The grip force controller may be configured to filter the signal and to determine the occurrence of the gripping event based on the filtered signal.

The minimum spread limit may be a negative spread. The grip force controller may be configured to reset the minimum spread limit on determining that any one or more of the following occur: the jaws are opened past an opening spread threshold; the demanded spread exceeds a demanded spread threshold; the demanded spread becomes positive; a measured spread becomes positive; the grip force applied between the jaws reduces past a further grip force threshold; a tension of a driving element or other component of the instrument reduces past a reset threshold tension; a torsion of a driving element or other component of the instrument reduces past a reset threshold torsion; a flexing of a driving element or other component of the instrument reduces past a reset threshold flexing; the jaws move relative to each other in the opening direction at a speed above an opening speed threshold; a commanded jaw movement in the opening direction exceeds a threshold commanded jaw movement; a change in force per unit of displacement exceeds a predetermined value; a grip force commanded by the surgeon decreases; a predetermined time, tᵣₑₛₑₜ, elapses; and an indication of an end of a gripping operation is received.

The grip force controller may be configured to determine that the instrument end effector is in a static condition where the average demanded motor force exceeds a static condition motor force. The determination that the instrument end effector is in a static condition may be made on determining the occurrence of the gripping event. The static condition motor force may be −180 units of effective force, and the average demanded motor force may exceed this when it becomes more negative than this value.

The grip force controller may be configured to: determine a yaw difference value based on a difference between a demanded yaw angle of the instrument end effector and a measured yaw angle of the instrument end effector; and determine that the instrument end effector is in a dynamic condition where it is determined that the yaw difference value is greater than a dynamic yaw value.

The grip force controller may be configured to determine that the instrument end effector is in a dynamic condition where it is determined that a yaw speed is greater than a dynamic yaw speed value. The grip force controller may be configured to determine that the instrument end effector is in a dynamic condition where the demanded motor force for one of the plurality of motors is greater than the motor force threshold and the demanded motor force for another of the plurality of motors is less than the motor force threshold.

Where it is determined that the instrument end effector is in a dynamic condition, the minimum spread limit for the jaws may be made more positive. The minimum spread limit may not be increased past a spread value corresponding to a minimum grip force threshold, thereby retaining a minimum grip force between the jaws. A spread-dependent gain may be applied to the demanded yaw angle.

The grip force controller may be configured to: determine a total force, F, on driving elements for driving each jaw; determine a rate of change of the total force, F, with spread of the jaws, S; determine a rate of change of spread of the jaws, S, with time, t; in dependence on the determined rate of change of the total force exceeding a predetermined constant, k_{S}, and the determined rate of change of spread of the jaws exceeding another predetermined constant, V_{S}, setting the instantaneous total force as a baseline force and setting the instantaneous spread as a baseline spread. The grip force controller may be configured to determine a change in total force relative to the baseline force, and to determine the occurrence of the gripping event where the determined change in total force exceeds a threshold force value. The grip force controller may be configured to reset the baseline force where it is determined that the spread of the jaws increases past the baseline spread and/or increases to a positive spread.

According to another aspect of the present invention, there is provided a method of controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the method comprising:
controlling movement of the jaws according to a demanded spread between the jaws;
determining an occurrence of a gripping event as the jaws are driven relative to each other in the closing direction so as to apply a grip force between the jaws;
determining a demanded spread between the jaws on determining the occurrence of the gripping event;
setting the determined demanded spread as a minimum spread limit for the jaws; and
controlling movement of the jaws using the minimum spread limit so as to control grip force applied by the jaws.

According to another aspect of the present invention, there is provided a grip force controller for controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the grip force controller being configured to:
control a force applied between the jaws according to a demanded force;
determine an occurrence of a gripping event as the jaws are driven relative to each other in the closing direction so as to apply a grip force between the jaws;
determine an indication of a demanded force between the jaws on determining the occurrence of the gripping event;
set the determined demanded force as a maximum demanded force limit for the jaws; and
control the force applied between the jaws using the maximum demanded force limit so as to control grip force applied by the jaws.

According to another aspect of the present invention, there is provided a method of controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the method comprising:
controlling a force applied between the jaws according to a demanded force;
determining an occurrence of a gripping event as the jaws are driven relative to each other in the closing direction so as to apply a grip force between the jaws;
determining an indication of a demanded force between the jaws on determining the occurrence of the gripping event;
setting the determined demanded force as a maximum demanded force limit for the jaws; and
controlling the force applied between the jaws using the maximum demanded force limit so as to control grip force applied by the jaws.

According to another aspect of the present invention, there is provided a grip force controller for controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the grip force controller being configured to:
control a force applied between the jaws according to a demanded force, and/or control movement of the jaws according to a demanded spread between the jaws;
determine an occurrence of a gripping event as the jaws are driven relative to each other in the closing direction so as to apply a grip force between the jaws;
determine an indication of a demanded force between the jaws and/or determine a demanded spread between the jaws on determining the occurrence of the gripping event;
where a demanded force is determined, set the determined demanded force as a maximum demanded force limit for the jaws, and where a demanded spread is determined, set the determined demanded spread as a minimum spread limit for the jaws; and
control the force applied between the jaws using the maximum demanded force limit and/or control movement of the jaws using the minimum spread limit so as to control grip force applied by the jaws.

According to another aspect of the present invention, there is provided a method of controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the method comprising:
controlling a force applied between the jaws according to a demanded force, and/or controlling movement of the jaws according to a demanded spread between the jaws;
determining an occurrence of a gripping event as the jaws are driven relative to each other in the closing direction so as to apply a grip force between the jaws;
determining an indication of a demanded force between the jaws and/or determining a demanded spread between the jaws on determining the occurrence of the gripping event;
where a demanded force is determined, setting the determined demanded force as a maximum demanded force limit for the jaws, and where a demanded spread is determined, setting the determined demanded spread as a minimum spread limit for the jaws; and
controlling the force applied between the jaws using the maximum demanded force limit and/or controlling movement of the jaws using the minimum spread limit so as to control grip force applied by the jaws.

According to another aspect of the present invention, there is provided a grip force controller for a surgical robotic system configured to perform a method as defined herein.

According to another aspect of the present invention, there is provided a surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, and a grip force controller configured to perform a method as defined herein.

According to another aspect of the present invention, there is provided a non-transitory computer readable storage medium having stored thereon computer readable instructions that, when executed at a computer system, cause the computer system to perform a method as defined herein.

Any feature of any aspect above can be combined with any one or more other feature of any aspect above. Any method feature may be rewritten as an apparatus feature, and vice versa.

### BRIEF DESCRIPTION OF THE DRAWINGS

The present invention will now be described by way of example with reference to the accompanying drawings.

In the drawings:

FIG. 1 schematically illustrates an example surgical robot performing an example surgical procedure;

FIG. 2 illustrates a surgical instrument;

FIG. 3 illustrates a surgical robot;

FIG. 4 illustrates a drive assembly interface of a surgical robot arm;

FIG. 5 illustrates an instrument interface of a surgical instrument;

FIG. 6 schematically illustrates an example operator console;

FIG. 7 illustrates an example of a rotatable jaw and associated driving elements;

FIG. 8 illustrates an example of an end effector having jaws and associated driving elements;

FIG. 9 schematically illustrates an example surgical robot system;

FIG. 10 illustrates an example of a change in overclose state;

FIG. 11 schematically illustrates the relationship between a drive force limit and a differential force limit;

FIG. 12 illustrates grip force changes over time for different control schemes;

FIG. 13 is a block diagram of an example method of controlling movement of jaws of an instrument; and

FIG. 14 illustrates components of an exemplary computing-based device.

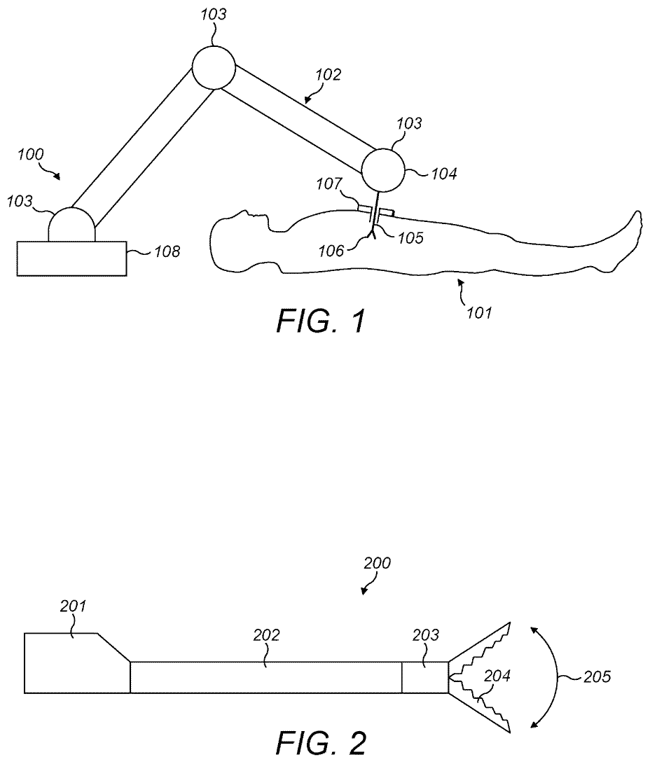

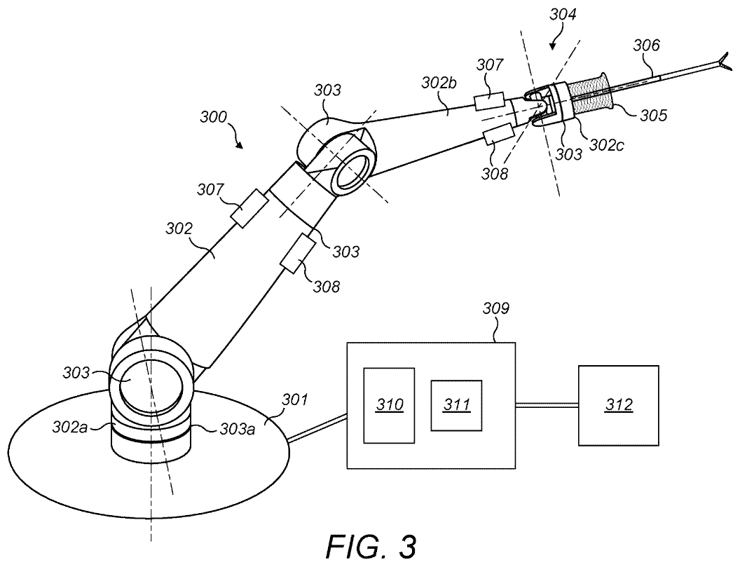

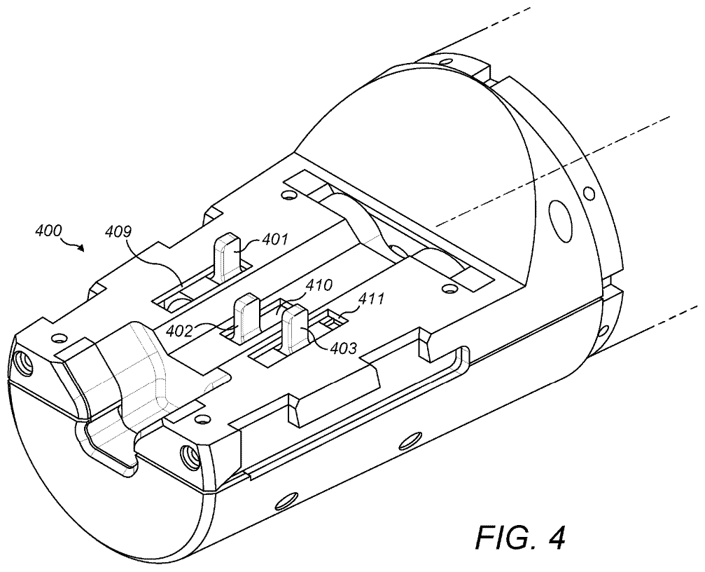

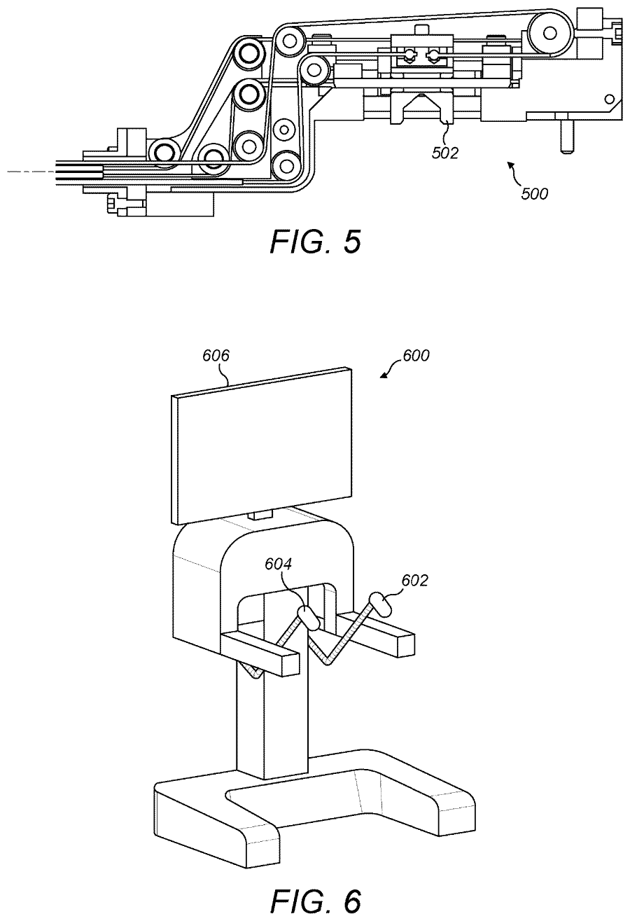

### DETAILED DESCRIPTION

The following description is presented by way of example to enable a person skilled in the art to make and use the invention. The present invention is not limited to the embodiments described herein and various modifications to the disclosed embodiments will be apparent to those skilled in the art. Embodiments are described by way of example only.

The following description describes the present techniques in the context of surgical robotic systems, though the features described below are not limited to such systems, but may be applied to robotic systems more generally. In some examples, the present techniques may be applied to robotic systems that operate remotely. Robotic systems can include manufacturing systems, such as vehicle manufacturing systems, parts handling systems, laboratory systems, and manipulators such as for hazardous materials or surgical manipulators.

The present inventors have found that there can be some variance, which is occasionally significant, in the force delivered by instrument jaws. The delivered force can depend upon the instrument used and/or the drive assembly actuating the instrument. This means that the same instrument used on two different arms, with different drive assemblies, can potentially deliver a significantly different maximum grip force. Likewise, different instruments coupled to the same drive assembly can also potentially deliver a significantly different maximum grip force.

The impact of this is that some instruments and arms (or combinations of instruments and arms) are better able to grip objects such as needles than others. From a user perspective, the difference in gripping capability is undesirable, and may appear like a system fault.

The present inventors have also appreciated that a further issue can be caused by one or more motors in the drive assembly saturating, i.e. reaching the maximum force that that motor can deliver, irrespective of the force demanded. At this point (once motor saturation has occurred), no additional force can be delivered by the instrument. This can lead to degradation in the gripping performance. The grip may become unstable. “Jerky” motion and grip forces may be observed in these situations.

At least some of these issues may stem from tolerances in the drive assembly, the arm/instrument interface and the instrument itself interacting with the position control of the instrument. Minor variations in manufacturing of some or all the components of these sub-systems have the potential to add up to an overall variability that has significant impact on the delivered force. For example, in some situations, component variability could lead to peak grip force delivered at the tip of the jaws, when a surgeon commands the jaws to grip with maximum strength, to vary from 10 N to 18 N.

The present inventors have further realised that this grip force variability is further complicated by the jaw spread angle of the instrument when maximum grip force is required. Since the size of an object being gripped can vary—for example different gauges of suture needle—and the position that the object is held in between the jaws also varies—i.e. nearer the pivot or nearer the tips—it is not known in advance at what angle the jaws will be when they need maximum grip on an object. Furthermore, variability in stiffness of the instrument will impact the required drive assembly travel (i.e. movement of a drive mechanism, such as a linear drive mechanism) to achieve a given grip force.

Hence, because of all the variables affecting grip force output, using a fixed relationship between an input controller (such as a hand controller pincer mechanism) and the jaw spread, across all instruments and drive assemblies, is unlikely to result in optimum control of the grip force.

It is therefore desirable to consider a new control scheme which is able to compensate for these inconsistencies. In the following, an adaptive gripping control scheme is described. This control scheme provides for a reactive control relationship between a hand controller and a drive interface. This control scheme therefore enables grip force output improvements, and consistency in force output. Advantageously, individual calibration of each instrument and drive assembly is not required.

In an exemplary control scheme, a two-stage mapping can be used between a commanded jaw position and an actual jaw aperture angle (a spread of the jaws). Suitably the mapping is linear. The threshold separating the two stages is preferably selected as the hand controller pince angle which should correspond to a 0 degree spread angle. As the pince angle continues to decrease, the jaws try to “overclose”, increasing the force being delivered. The increase in the force being delivered is suitably linear. The first stage can have a lower gradient than the second stage so that the grip force ramps up when the jaws are overclosed.

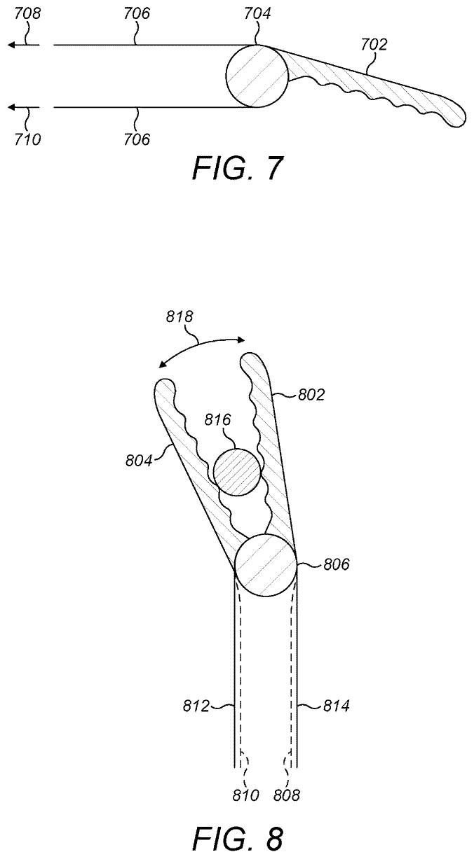

Yaw tracking may also cause issues in delivering and maintaining grip force. When the control system demands maximum grip, the drive assembly can respond by actuating drive motors or actuators to pull both jaws together hard. The jaws can be driven via driving elements which couple to the drive assembly, through an instrument interface, and to the jaws. Suitably the driving elements are cables such as metal cables. This is illustrated in FIG. 7. FIG. 7 shows a single jaw 702 (for clarity) which is rotatable about a pulley 704. The jaw 702 can be unitary with the pulley 704. For example, a pulley channel can be formed in a proximal part of the jaw 702. A driving element 706 can run over the pulley 704 to control rotational movement about the pulley axis of the jaw 702. A single driving element can loop over the pulley 704, or multiple driving elements can be provided. Where multiple driving elements are provided, a first driving element can run over part of the pulley 704 in a first direction about the rotational axis of the pulley, and can attach to a proximal part of the jaw 702. A second driving element can run over another part of the pulley 704 in a second direction about the rotational axis of the pulley, and can attach to the proximal part of the jaw 702 (this can be, but need not be, at the same location as the attachment of the first driving element). Pulling the single driving element 706 in one direction 708 (or pulling, e.g., the first driving element) can cause the jaw to rotate in an opening direction. Pulling the single driving element 706 in the other direction 710 (or pulling, e.g., the second driving element) can cause the jaw to rotate in a closing direction.

Reference is now made to FIG. 8, illustrating an object gripped between two jaws. In FIG. 8, a first, right-hand, jaw 802 and a second, left-hand, jaw 804 are rotatably coupled to a pulley 806. A first opening driving element 808 is coupled to the right-hand jaw 802. A first closing driving element 810 is coupled to the right-hand jaw 802. Pulling the first opening driving element 808 will cause the right-hand jaw 802 to move in an opening direction (i.e. away from the left-hand jaw 804). Pulling the first closing driving element 810 will cause the right-hand jaw 802 to move in a closing direction (i.e. towards the left-hand jaw 804). A second opening driving element 812 is coupled to the left-hand jaw 804. A second closing driving element 814 is coupled to the left-hand jaw 804. Pulling the second opening driving element 812 will cause the left-hand jaw 804 to move in an opening direction (i.e. away from the right-hand jaw 802). Pulling the second closing driving element 814 will cause the left-hand jaw 804 to move in a closing direction (i.e. towards the right-hand jaw 802). An object 816 is gripped partway along the jaws 802 804. The first opening driving element and the first closing driving element are shown in dashed lines to help distinguish them from the second opening driving element and the second closing driving element. The dashed lines are shown as being laterally offset from the solid lines merely for clarity. The pulleys about which the driving elements pass are suitably of the same size, although they could be different.

Pulling the jaws together hard can stretch the driving elements (e.g., with reference to FIG. 8, the first closing driving element 810 and the second closing driving element 814) on the tension side on both jaws. However, if the end effector is moving as well as gripping—specifically moving in yaw (see arrow 818 in FIG. 8)—then one driving element is being pulled hard and the other is being pulled less hard. In fact, the jaw coupled to the driving element that is being pulled less hard is being (or at least could be) back-driven by the other jaw coupled to the driving element that is being pulled hard. For example, where the end effector illustrated in FIG. 8 is moving in yaw to the left, whilst gripping, the first closing driving element 810 will be pulled harder than the second closing driving element 814. Tension applied to the first closing driving element 810, causing the right-hand jaw 802 to move in the closing direction (i.e. to the left in FIG. 8) can drive the left-hand jaw 804 to the left as well (which is the opening direction in respect of this left-hand jaw 804).

Moving the end effector in the yaw direction whilst gripping may also give rise to inconsistent grip performance. The reason for this is that in the non-moving case, both cables stretch by the same amount. However, in the yawing case, one cable stretches more than the other. The situation where the end effector is not moving while gripping can be referred to as a “static” condition and the case where the end effector is in motion while gripping can be referred to as a “dynamic” condition.

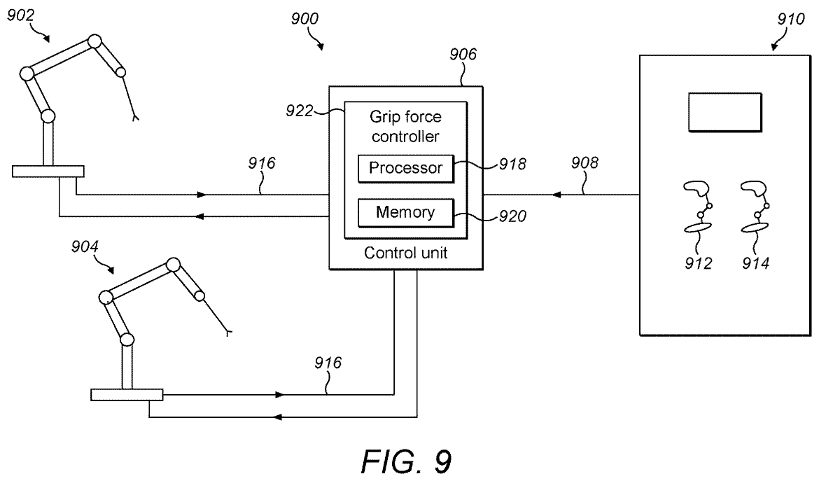

The control scheme discussed herein is able to compensate for grip variations in both static and dynamic conditions, as explained further elsewhere herein. Reference is now made to FIG. 9 which illustrates an example surgical robotic system 900. In this example the surgical robotic system 900 comprises two surgical robots 902 and 904 driven by a control unit 906. The control unit 906 receives inputs 908 from an operator console 910 (such as, but not limited to the operator console 600 of FIG. 6), including inputs from first and second hand controllers 912, 914. The control unit 906 may receive other inputs from the operator console 910, such as foot pedal(s) inputs, voice recognition inputs, gesture recognition inputs, eye recognition inputs etc. The control unit 906 also receives inputs 916 from the surgical robots 902, 904. These inputs include sensor data from position sensors and torque sensors located on the robot arm joints. The control unit 906 may receive other inputs 916 from each robot, such as force feedback, data from or about the surgical instruments etc. The control unit 906 drives the robots 902, 904 in response to the inputs it receives from the robots 902, 904 and the operator console 910. The control unit 906 comprises one or more processors 918 and a memory 920. The memory 920 stores, in a non-transient way, software code that can be executed by the one or more processors 918 to control the drivers. The control unit 906 may embody or comprise a grip force controller. In FIG. 9, the control unit 906 is shown as comprising the grip force controller 922. In this example, the grip force controller 922 comprises the processor 918 and the memory 920.

While the example surgical robotic system 900 of FIG. 9 comprises two surgical robots, it will be evident to a person of skill in the art that the methods and techniques described herein are equally applicable to surgical robotic systems with only one surgical robot and surgical robotic systems with more than two surgical robots.

One possible solution to address the above issues is to individually calibrate all the separate sub-systems and then modify control parameters when these calibrated sub-systems are brought together on the arm to compensate for the differences. However, this approach is undesirable, because it not only means that the calibration needs to be done, but also that the values need to be stored following the calibration. These stored values then need to be communicated to the control system in some way, and then used by the control system in the control of the sub-systems. This approach would be challenging because, at the point of manufacture, it is not known where each sub-system will end up, or which other sub-system it will be used with. Also, this approach of carrying out a calibration process then storing calibration parameters is likely to require an additional setup step for surgical teams, which is undesirable. Further, the differences in jaw angle described above (e.g. where holding a needle at different locations along a length of an end effector) also make this approach difficult. What would be likely to be needed is a full characterisation of all the components rather than a (relatively simpler) calibration. In addition, with this approach, where the master-slave control relationship is fixed for the duration of the surgery, the system would not be able to properly address issues arising from differences in the size of an object being grasped.

The present inventors have realised that what is needed is an “on-the-fly” method to compensate for the variability. It is therefore desirable to provide a control scheme that does not require:
Storing calibration parameters of individual components at the point of manufacture;
Additional steps by the surgical team at setup or during a procedure; or
Knowledge of the position in the jaws or size of the object being grasped.

Logically, the point in time where these can be avoided is at the point of grasping an object. It is therefore appropriate to consider how to determine when the instrument is grasping an object. There are several ways in which this can be done. One approach, which is currently the preferred approach, is to use the demanded motor force (or torque). It is also possible to use one of the following: grip force, driving element tension, and visual servoing. More than one of these approaches can be used together. For example, an exemplary approach can determine that an object is being grasped using the demanded motor force and the driving element tension. Other combinations of two or more of these approaches are also possible.

The control scheme presented herein is useful for any gripping instrument, e.g. a jawed instrument that has a plurality of facing jaws that can be brought together to grip an object and separated to release the grip on an object. An example of such an instrument is a needle holder instrument. For completeness, it is noted that the instrument can be in a gripping condition where the jaws are touching one another. This could be because the instrument is gripping a very thin (and/or compressible) object or because the jaws have been closed without an object in between the jaws. The control scheme described herein is applicable whether or not an object is held between the jaws as the jaws are closed together. The control scheme is useful where the jaws meet resistance on closing and a grip force is applied between the jaws.

The preferred control scheme dynamically adjusts the demanded overclose of an instrument when the instrument is gripping to account for a variation in the zeroing and/or stiffness of the mechanical system. The control scheme uses thresholds in units of demanded drive force from motors or actuators in the drive assembly that are controllable to effect movement of the jaws of the instrument. The control scheme can ‘clip’ a maximum overclose or overspread (e.g. a commanded negative jaw spread) value, to correspond to a threshold force of the motor, each time a jaw closing event/gripping event occurs. The control scheme is particularly useful in high-force jaw closing or gripping events. In such events, it is possible that motors may reach saturation, or driving element stretch may occur, for example.

The maximum overspread is the most negative spread that can be demanded. Physically, the jaws cannot go past 0 degrees of spread, i.e. the jaws cannot physically have a negative spread. However, the demanded spread (or simply ‘spread’ as used herein) can be used as a measure of the demanded grip force; that is, the spread of the jaws may be considered to correspond to force, such as commanded force, on or between the jaws. A more negative demanded spread can be indicative of a greater demanded grip force. Note that in examples where an object such as a needle is gripped between the jaws, there will be some positive value of spread below which the jaws cannot close. Thus, an overclose condition may apply even where the jaws have a positive spread.

The maximum overspread can be limited, e.g. to a minimum spread limit (which is suitably a spread, either positive or negative, which is the most negative spread that can be demanded by the control system) when it is estimated that the jaws are gripping something hard. The overclose limit can be equal to the demanded spread of the jaws at the time when a maximum demanded force threshold is met.

A grip force controller for controlling grip force of an instrument in a surgical robotic system can be provided in accordance with the techniques discussed herein. The grip force controller may be located at the control unit 906 illustrated in FIG. 9. The grip force controller 922 is suitably configured to control movement of jaws 802, 804 of an instrument 200 according to a demanded spread between the jaws. The demanded spread can be determined in response to inputs received at a hand controller 912, 914. The demanded spread may be determined at the control unit 906, for example at the grip force controller 922. The grip force controller 922 is configured to determine an occurrence of a gripping event as the jaws 802, 804 are driven relative to each other in the closing direction. As the jaws are driven relative to each other in the closing direction, a grip force can thereby be applied between the jaws. The grip force controller 922 is configured to determine a demanded spread between the jaws on determining the occurrence of the gripping event. That is, when it is determined that the gripping event occurs, the grip force controller can determine the demanded spread between the jaws at the time that the gripping event occurs. The grip force controller is configured to set the determined demanded spread as a minimum spread limit for the jaws. This setting of the determined demanded spread as the minimum spread limit for the jaws means that the jaws will not thereafter be controlled such that a more negative spread is demanded by the control system. The grip force controller is configured to control movement of the jaws using the minimum spread limit so as to control grip force applied by the jaws. In other words, controlling the jaw movement (or controlling the end effector) can be performed such that the control system does not demand a grip force greater than the grip force at the time that the gripping event occurred.

The grip force controller 922 can be configured to determine the occurrence of the gripping event on determining that any one or more of the following occur:
a grip force between the jaws 802, 804 exceeds a grip force threshold.
The grip force can be considered to exceed the grip force threshold when the grip force becomes larger than the grip force threshold. The grip force may be obtained from measurements at the jaws, for example using one or more sensors arranged to sense the grip force. The sensor(s) may comprise a strain gauge. The sensor(s) may be located at the jaws, or remote from the jaws and configured to sense the force at the jaws, for example by sensing force transmitted to the jaws. The sensor(s) may sense deformation of components of the instrument, including for example deformation of components of the instrument other than the jaws.
a demanded motor force (or torque) of a motor of the drive assembly for transferring drive to one of the jaws exceeds a motor force threshold.
The demanded motor force can be considered to exceed the motor force threshold when a positive demanded motor force becomes larger (more positive) than the motor force threshold. Equivalently, it is possible to define motor force in the closing direction as negative, in which case the demanded motor force can be considered to exceed the motor force threshold when a negative demanded motor force becomes smaller (more negative) than the motor force threshold.
an average demanded motor force (or torque) of a plurality of motors of the drive assembly for transferring drive to respective ones of the jaws exceeds an average motor force threshold.
The average demanded motor force can be calculated as the mean of the individual demanded motor forces. The average demanded motor force can be considered to exceed the average motor force threshold when a positive average demanded motor force becomes larger (more positive) than the average motor force threshold, or when a negative average demanded motor force becomes smaller (more negative) than the average motor force threshold.
the motor of the drive assembly for transferring drive to one of the jaws approaches or reaches a saturation point.
The motor can reach a saturation point where it is no longer able to increase force output. The motor can approach the saturation point where it is within a predefined proportion of the possible force output, for example, where the motor is supplying at least 95% of the possible motor output, or at least 90% of the possible motor output. Other setpoints of the possible motor output can be selected as desired. Where force output may be demanded to increase quickly at relatively higher forces, it may be appropriate to select a lower setpoint to avoid reaching the saturation point.
a current through the motor of the drive assembly for transferring drive to one of the jaws exceeds a current threshold.
The current through the motor can considered to be representative of the motor force output, and/or of the saturation point of the motor.
a driving element of the instrument, by which drive is transferred to one of the jaws, approaches or reaches a physical limit of the driving element.
The driving element suitably comprises a flexible driving element, such as a cable. The driving element may comprise or be formed from metal, or braided metal fibres. The driving element may comprise a rod. The physical limit of the driving element may be a tension in the driving element above which plastic deformation of the driving element occurs. The physical limit of the driving element may be a tension in the driving element above which the cable is liable to fray or snap. The physical limit of the driving element may be a torsion in the driving element above which plastic deformation of the driving element occurs.
Tension in the driving element can be determined using one or more load cells. Suitably, a triple load cell is used. Load cell measurements may be susceptible to change with environmental conditions, such as temperature. To reduce environmental effects on load cell measurements, suitably relative measurements of the load cell are used. It is also possible to use absolute measurements from a load cell, which can optionally be filtered, for example by a low pass filter.
a physical limit is reached (or a threshold limit is reached or exceeded) of a component of the instrument, for example of the instrument end effector.
The component may comprise a jaw supporting component, e.g. a yoke and/or an end of a shaft in the end effector. The component may comprise the instrument shaft. A physical limit for the component can be considered to be reached where the tension and/or torsion and/or flexing of the component causing plastic deformation of the component is exceeded. A threshold limit can be set, e.g. for the tension and/or torsion and/or flexing of the component, which is below the plastic deformation limit. The gripping event can be determined to occur when such a threshold limit is reached or exceeded. Setting the threshold limit to be less than the plastic deformation limit enables undesirable plastic deformation of the component to be avoided.

The grip force controller can be configured to determine the occurrence of the gripping event based on a signal indicative of one or more of:
the grip force between the jaws.
The gripping event can be determined when the signal indicates that the grip force exceeds the grip force threshold. The grip force can be a commanded grip force or a measured grip force.
the demanded motor force of the motor.
The gripping event can be determined when the signal indicates that the demanded motor force exceeds the motor force threshold.
the average demanded motor force of the plurality of motors.
The gripping event can be determined when the signal indicates that the average demanded motor force exceeds the average motor force threshold.
motor saturation of the motor of the drive assembly for transferring drive to one of the jaws.
The gripping event can be determined when the signal indicates that the motor saturation approaches or reaches the saturation point.
current through the motor of the drive assembly for transferring drive to one of the jaws. The gripping event can be determined when the signal indicates that the current through the motor exceeds a current threshold.
tension in the driving element of the instrument by which drive is transferred to one of the jaws.
The gripping event can be determined when the signal indicates that the tension in the driving element approaches or reaches the physical limit of the driving element. For example, the gripping event can be determined when the signal indicates that the tension in the driving element exceeds a threshold tension.

The signal can comprise or be derived from visual servoing based on an image of the instrument. Suitably, the image field of view includes the instrument end effector. This enables visual analysis of the jaws, e.g. of the position and movement of the jaws, of the end effector to be performed. In some implementations, the signal can comprise or be derived from visual servoing of an image of the hand controller 912. In this case, the image field of view will suitably include the hand controller pincer. This enables visual analysis of the pincer (e.g. position and movement) to be performed. Visual servoing can comprise analysing visual feedback, using e.g. visible light or infrared light. The visual servoing may comprise obtaining images using a thermal camera. The visual servoing suitably comprises applying image processing algorithms to images obtained of the instrument and/or of the hand controller.

As jaw closing motion is performed, resulting in a reducing spread between the jaws, the demanded motor force can be monitored. The demanded spread is also suitably monitored. Optionally, the demanded motor force can be filtered, for example to remove noise. The filtering suitably increases the signal to noise ratio of a signal indicative of the demanded motor force. For example, the demanded motor force can be filtered using a low pass filter. Once a threshold motor force or demanded motor force is reached, the demanded spread at that time is set as the maximum allowed overclose (or maximum negative spread), e.g. a minimum spread limit. The minimum spread limit may be negative, e.g. it may be indicative of an overclose condition.

The grip force controller is suitably configured to reset the minimum spread limit on determining that any one or more of the following occur:
the jaws are opened past an opening spread threshold.
The opening spread threshold is suitably a spread value that indicates that the high-force conditions are no longer present. For example, the opening spread threshold may be defined in dependence on the minimum spread limit. The opening spread threshold may be determined as the minimum spread limit plus a predefined spread angle, for example 2 degrees, 5 degrees, or 10 degrees. The opening spread value may be a fixed value, for example a positive spread of 2 degrees, 5 degrees, 10 degrees or 15 degrees. Higher spread angles can be selected. The opening spread angle may be selected in dependence on an object likely to be gripped by the instrument. For example, where a needle of a given thickness is to be gripped, the jaw spread on gripping the needle will depend on where along the length of the jaw the needle is gripped. The opening spread threshold may be set at an angle which is sufficient to release the needle wherever it is held within the jaws.
the demanded spread exceeds a demanded spread threshold.
The demanded spread threshold may take values as indicated above for the opening spread threshold. Other values may be selected for the demanded spread threshold.
the demanded spread, and/or a calculated or measured spread, becomes positive.
the grip force applied between the jaws reduces past a further grip force threshold.
The grip force may be a demanded grip force or a measured grip force. Where the grip force reduces past the further grip force threshold, this can indicate that the jaws are not gripping anything with a high enough force to cause the force variations discussed herein. The further grip force threshold may be the same as the grip force threshold (at which the gripping event can be determined). The further grip force threshold may be lower than the grip force threshold. This approach can be considered to introduce a hysteresis into the control system. The control system can thereby operate in a more stable manner.
a tension and/or torsion and/or flexing of a driving element or other component of the instrument reduces past a reset threshold tension, a reset threshold torsion or a reset threshold flexing, respectively.
Where the tension, torsion and/or flexing reduces past the respective threshold, this can indicate that the jaws are no longer exerting a gripping force high enough to cause the force variations discussed herein. Where the tension, torsion and/or flexing reduces past the respective threshold, this can be indicative of a reduction in the stress/strain within the system. The reset threshold tension, the reset threshold torsion and/or the reset threshold flexing may be the same as the threshold limit for the tension, torsion and/or flexing, respectively (at which the gripping event can be determined). The reset thresholds may be lower than the threshold limits. This approach can be considered to introduce a hysteresis into the control system. The control system can thereby operate in a more stable manner.
the jaws move relative to each other in the opening direction at a speed above an opening speed threshold.
Movement of the jaws in an opening direction, relative to one another, can be indicative of a desired relaxation in the grip. Movement of the jaws can, e.g., be determined by visual means and/or by tracking the position of the instrument drive mechanisms. The opening speed threshold may comprise a speed and a time for which that speed is maintained. For example, the opening speed threshold may comprise a speed greater than 0 rad/s, which is maintained for a time (t) greater than tₘᵢₙ. Thus, the opening speed threshold may be met when a positive speed occurs for a time greater than tₘᵢₙ.
a commanded jaw movement in the opening direction exceeds a threshold commanded jaw movement.
The jaws may not move even when the grip is being relaxed. It is possible for the jaws to remain stationary, but for the force between the jaws to reduce. Thus, a reduction in the jaw gripping force can be determined where the drive mechanisms of the drive assembly (and/or the instrument drive systems to which the drive assembly is coupled) move in the opening direction, e.g. where the drive mechanisms move by at least 0.5 mm in the opening direction.
a change in force per unit of displacement exceeds a predetermined value.
The force per unit of displacement can provide a measure of the stiffness of the system. Determining the change in force per unit of displacement can indicate a release of elastic energy, e.g. in the drive assembly, as the jaws start to open.
the grip force commanded by the surgeon decreases.
It is noted that the present techniques are useful to provide a stable grip force in a position control scheme, but it should also be noted that it would be possible to apply the present techniques to a force control scheme. For example, for a force controller with a force sensor, such as at the jaw, it may be useful to determine high force events and grip thresholds to compensate for build variations.
a predetermined time, tᵣₑₛₑₜ, has elapsed.
an indication of an end of a gripping operation is received.
The indication may be provided by a surgeon. The indication may be determined from motion of the instruments of the robotic system, e.g. a second instrument approaches to take a needle from a first instrument.

The concepts of static grip and dynamic grip have been introduced elsewhere herein. Static grip relates to a condition where the end effector is not moving (or not appreciably moving) in a yaw direction whilst gripping. Dynamic grip relates to a condition where the end effector is moving in a yaw direction whilst gripping. In the dynamic condition, yaw tracking errors can arise. To achieve more accurate yaw tracking, it is possible, in the dynamic condition, to relax the control conditions, e.g. to permit a different minimum spread limit for the jaws. The minimum spread limit for the jaws can be increased (made more positive). This means that the jaws will grip with slightly less force, but it also enables a more consistent grip. This modification can help account for stretch of driving elements, and can thereby increase accuracy. In some implementations, the minimum spread limit in the dynamic case differs by approximately 25% compared to the minimum spread limit in the static case. Spread is suitably indicated in radians, and can be within a range extending to −2.5 rad. The minimum spread limit for the jaws may be in the range −2.5 rad to −2 rad for the static case. The minimum spread limit may be in the range −2 rad to −1.5 rad for the dynamic case. As will be understood from the present specification, although spread can imply an opening angle between the jaws, it is also indicative of commanded force applied at the jaws. In some implementations, spread may therefore be specified in other units, for example in terms of force (N) or tension.

The static condition is identifiable by detecting that both motors of a drive assembly for driving the jaws of the instrument are demanding a high force (i.e. when the demanded motor force exceeds a static condition motor force threshold, e.g. a proportion of a total possible grip force, such as 85% of the total possible grip force; this proportion can be selected as desired, but suitably will take into account the motor rating in the particular high temperature and humidity conditions expected during surgery; a safety factor may be applied to the proportion. This proportion may be determined based on a lifetime condition for the instrument (e.g. a lower proportion will generally lead to the output of lower grip forces but will ensure the usable life of the instrument is extended). The proportion may be determined empirically as a value at which the force delivered by the instrument starts becoming unstable). Where both motors are demanding a high force, it is likely that both the jaws are being pulled hard in their respective closing directions. The static condition motor force may be in the range −150 units of effective force to −200 units of effective force, for example approximately −180 units of effective force. The ‘units of effective force’ may correspond to Newtons. The force as measured in the units of effective force may be a force corresponding to a known current through a drive assembly motor leading to the force (or torque). Negative force is used here because the jaws are closing. The static condition may be identifiable on determining the occurrence of the gripping event.

The dynamic condition is identifiable by detecting a yaw tracking error. A yaw difference value can be calculated, for example at or by the grip force controller 922, as the difference between a demanded yaw angle of the instrument end effector and a measured yaw angle of the end effector. It can be determined that the instrument is in the dynamic condition where the yaw difference value is greater than a dynamic yaw threshold value. The dynamic yaw threshold value can be set at any desired value, for example in the range 0.01 rad to 0.1 rad, such as 0.05 rad. Where the yaw difference value exceeds the dynamic yaw threshold value, it is likely that the end effector is moving (or is being commanded to move) in a yaw direction whilst gripping.

The dynamic condition may also be identifiable by determining (e.g. at or using the grip force controller) that a yaw speed (or commanded yaw speed) of the end effector is greater than a dynamic yaw speed threshold. The dynamic yaw speed threshold is suitably in the range 0.3 rad/second to 1.7 rad/second, or in the range 0.5 rad/second to 1.5 rad/second, e.g. approximately 1 rad/second.

The dynamic condition may also be identifiable by determining (e.g. at or using the grip force controller) that the demanded motor force for one of the plurality of motors is greater than the motor force threshold, and the demanded motor force for another of the plurality of motors is less than the motor force threshold, and the difference between the two demanded motor forces is above a predefined proportional difference. In an example, the static condition can be determined where the average demanded motor force exceeds −180 units of effective force. In the static condition, both jaws are being pulled hard. In the dynamic condition, one jaw is being pulled harder than the other jaw. This can be identified by determining that the demanded motor force driving one jaw is, e.g., more than 1.5 times, or more than 2 times, or more than 3 times the demanded motor force driving the other jaw.

The dynamic condition may be identifiable by determining (e.g. at or using the grip force controller) that the demanded motor force for one of the plurality of motors acts in the same direction as the demanded motor force for another of the plurality of motors. For example, it can be determined that the right-hand jaw is moving to the left in the yaw direction (in a closing direction for this jaw) and that the left-hand jaw is moving to the left in the yaw direction too (in an opening direction for this jaw). Whether the jaws are opening or closing relative to one another will depend on the relative motor forces driving each jaw, or on the relative jaw speeds. In this example, where the right-hand jaw is being driven by a greater motor force (or at a greater speed) than the left-hand jaw, the jaws will close. Where the right-hand jaw is being driven by a lesser motor force (or at a lower speed) than the left-hand jaw, the jaws will open.

In some implementations, where conditions indicating a yaw tracking error are met, the minimum spread limit can be increased (made more positive). Making the minimum spread more positive in this way can have the effect of relaxing the grip force applied by the end effector. Making the minimum spread limit more positive can enable a greater yaw movement of the end effector. It can enable this greater yaw movement by relaxing the restrictions caused by the minimum spread limit in the control of the end effector. The minimum spread limit can be increased by a predetermined amount (e.g. by 25%, or another suitable amount, which may depend on characteristics of the system, and/or on the yaw tracking error), or gradually increased until a new gripping event is determined.

Suitably, the minimum spread limit is not increased past a spread value corresponding to a minimum grip force threshold, thereby retaining a minimum grip force between the jaws. This means that the grip can be maintained, and an object held between the jaws, such as a needle, will not be accidentally dropped or released.

If certain conditions, e.g. indicating a yaw tracking error, are met, then the minimum spread limit can be made more positive (i.e. grip force can be relaxed). These conditions comprise:
A yaw tracking error is built up (e.g. of at least 0.05 rad).
The absolute value of the commanded yaw speed is above a given value, e.g. 1 rad/second.
The minimum demanded (negative) motor force (or filtered demanded motor force) for the motors or actuators in the drive assembly is less than the demanded motor force (or filtered demanded motor force) that is indicative of the gripping event. This can mean that one of the drive forces or motors is near saturation.

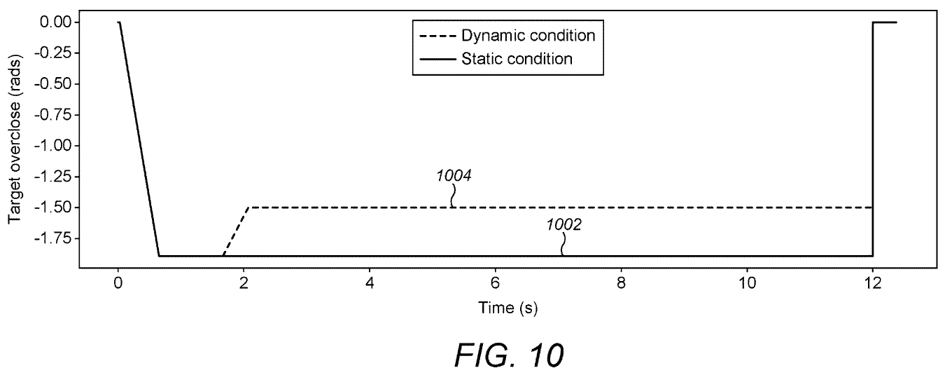

FIG. 10 illustrates an example of a change in overclose state (e.g. a change in minimum spread limit) as the control system transitions from static to dynamic control, i.e. as the end effector transitions into moving in the yaw direction. The lower plot 1002 represents the initial minimum spread limit (in the static condition). Where the dynamic condition is taken into account, the minimum spread limit can change to become more positive. This is indicated in the upper plot 1004, which shows a change to a more positive minimum spread limit (or target overclose value) following a transition to dynamic control which occurs, in this example, just before 2 seconds as indicated on the x-axis.

In some implementations, two motors or actuators of the drive assembly control movement of the jaws so as to control both the spread and yaw. In some cases, when the instrument is at a yaw limit and further spread is demanded, spread can be prioritised over yaw. That is, to achieve a demanded spread, the control system can accept an error (or a greater error) in yaw. Since the desired position of the motors or actuators driving the jaws is affected by the demanded yaw and the demanded spread, the more grip that is demanded the bigger yaw error that may result.

In one approach, in order to minimise the yaw tracking error, a spread-dependent gain can be applied to the demanded yaw angle (θ_{y}). This has the effect of increasing the demanded yaw angle where the spread is increased. Hence, a larger yaw angle is demanded which can more closely match

$$β = \{ \begin{matrix}1 , & θ \text{?} \geq 0 , or instrument is not needle holder \\ 1 - γ θ \text{?} & θ \text{?} < 0 , and instrument is needle holder\end{matrix}$$

$$\text{?} \text{indicates text missing or illegible when filed}$$

for γ=0.055 rad⁻¹.

the yaw angle as determined from the positions of the motors as they control the demanded spread. For a relatively large (and negative) demanded spread, a gain (B) greater than 1 can be used, which can result in an enhanced yaw angle (θ′_{y}). By enhancing the yaw angle proportionally to the demanded spread, the effect that demanding a large negative spread has on the yaw tracking error can be at least partially cancelled out. At least partially cancelling out this effect can help avoid unwanted and/or unexpected motions, where the control system is having to catch up with the commanded motions. In one example application of this approach, the calculations performed are the following:

θₛ represents the demanded spread angle.

The individual joint jaw angles (θ_{y1} and θ_{y2}) can then be calculated as:

$${θ}_{y 1} = {θ}_{y}^{′} + \frac{θ \text{?}}{2}$$

$${θ}_{y 2} = {θ}_{y}^{′} - \frac{θ \text{?}}{2}$$

$$\text{?} \text{indicates text missing or illegible when filed}$$

As discussed herein, force variability can be due to the manufacturing differences in one or more sub-system. Force variability can also arise from assumptions on the interface calibration (e.g. of the interface between the drive assembly and the instrument). Variability in forces can lead to poor grip force repeatability from a single drive assembly (e.g. from one or both motors of the drive assembly that are configured to actuate the jaws), and/or poor grip force reproducibility across multiple drive assemblies, and/or larger-than-intended forces being applied to instruments such as needle holders which can cause premature failures in the instruments. It is important in instrument control that drive assembly motor current saturation and peak output force are carefully controlled to improve dynamic performance and instrument lifetime.

A further optional modification to the control scheme implemented by the grip force controller (or elsewhere in the control system), as will now be described, may compensate for differences in the calibration between different robot interfaces. The interfaces may be calibrated slightly differently, or their calibration may differ from their actual performance. Hence it can be important to tune the relationship between the force delivered by the motors and the desired force in a particular use case.

Load cell force readings, such as triple load cell force readings, may be used, which enable measurement of tension in a driving element, such as a cable. Such force readings may have thermal offset issues. It is therefore useful if differential rather than absolute force readings are considered. Absolute force readings may, however, still be used. Where absolute force readings are used, the absolute force readings may be filtered. The absolute force readings may be filtered by a low pass filter. It is preferable to use differential force readings to improve accuracy. The differential force readings may also be filtered, for example using a low pass filter.

The term “motor force” is used herein. In the examples discussed herein, the instrument drive is linear. In general, the output from a motor can be described as a torque. It will be understood that references to motor forces herein are equally applicable to motor torques.

As described herein, the transition into the state where the minimum spread limit is set, i.e. on detection of the gripping event, can be controlled in dependence on the minimum demanded drive force (which can optionally be low pass filtered) on the drive assembly motors or actuators that are configured to drive the jaws in the yaw direction, which can be referred to as m1 and m2. The minimum demanded drive forces can be represented as (min (τₘ₁, τₘ₂)). The transition into this state can occur on determining that (min (τₘ₁, τₘ₂)) is below (e.g. more negative than) a threshold value (e.g. −180 effective force units).

In some implementations there may be a second condition that if met would also result in entry to this state where the minimum spread limit is set. The second condition is as follows. During closing motion of the jaws:
1. The input force, F, (e.g. the triple load cell force readings) (F=fₘ₁+fₘ₂) and the measured instrument spread(S) can be set as baselines (e.g. respectively as a baseline force and a baseline spread), when both of the following conditions are met:
   a. The stiffness calculated using the input force (F=fₘ₁+fₘ₂) first exceeds a threshold. This provides a measure of the resistance against closing the jaws. A greater stiffness will indicate that the jaws are closed and cables are being stretched. This condition can be represented as:

dF/dS>k_{S}
where k_{S} is a predefined value.
b. The jaws of the instrument are closing at a rate faster than a threshold rate (Vₛ), where the spread and threshold are both negative. This indicates how much the surgeon is willing to grip. When the surgeon wants to grip hard, the pincer will be closed fast, and the commanded spread will quickly decrease too. This condition can be represented as:

$$dS / dt < {V}_{S}$$

2. If the change in input force relative to the baseline force exceeds a threshold, fₜₕᵣₑₛₕ,

ΔF<fₜₕᵣₑₛₕ
(where the change in force is negative due to being in the closing direction), the system can be configured to enter the state where the minimum spread limit is set. This may indicate that the tension in the driving element is at an expected value, such as a threshold value indicating that grip force control is desirable, or that the tension matches an expected tension for the demanded grip force.

The baseline force is suitably reset when the jaws are opened beyond the baseline spread. The baseline force can be reset when the jaws are opened beyond zero spread. Resetting the baseline force can comprise removing the baseline force (so that a baseline force value no longer exists in the system) or setting the baseline force to zero or to another predefined or user-selectable value.

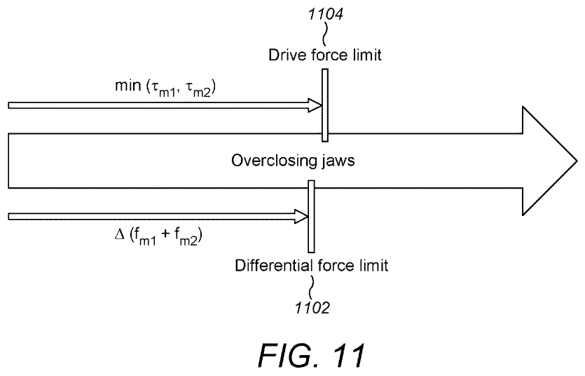

Conceptually, the modified control scheme, using this second condition, can be thought of as having two competing terms to determine the overclose limit for the instrument. FIG. 11 represents this schematically. It is desirable to set the differential force limit 1102 (e.g. as determined using the second condition) such that it is triggered more often than the drive force limit 1104 (e.g. as determined using the demanded motor force or torque, as discussed elsewhere herein; measurements of driving element tension, e.g. using triple load cell readings could additionally or alternatively be used to determine the drive force limit). This is because the drive force limit can act as a backstop to help ensure good dynamic performance by protecting against motor saturation whereas the differential force limit can more precisely control the differential force input to the instrument and thus the tension and grip force generated.

The control scheme discussed herein, in particular the modified control scheme, can provide the following benefits to controlling a jawed instrument such as a needle holder.
Driving element protection: The limit on maximum change in force from the baseline force should help prevent excessive tension being applied to the driving elements. It may also counteract any temporal or spatial variability in the interface calibration, e.g. drift in motor output which can lead to the application of forces varying from the demanded forces.
Repeatable and reproducible grip force: As grip force is proportional to the change in force from the baseline force, the control scheme can help ensure that the drive assembly motors more consistently deliver a fixed drive force to the instrument, leading to a more consistent grip force.
Equivalent dynamic performance: As, in the modified control scheme, the existing drive force entry condition is maintained, it will act as a backstop helping ensure motor saturation is avoided. This can thereby help ensure that the ‘gripping whilst yawing’ performance is maintained.

The control scheme, and grip force controller, discussed herein help ensure that consistent grip performance is achievable with different instruments and/or with different drive assemblies. In accordance with the present techniques, this consistent performance is achievable without having to perform additional calibration actions.

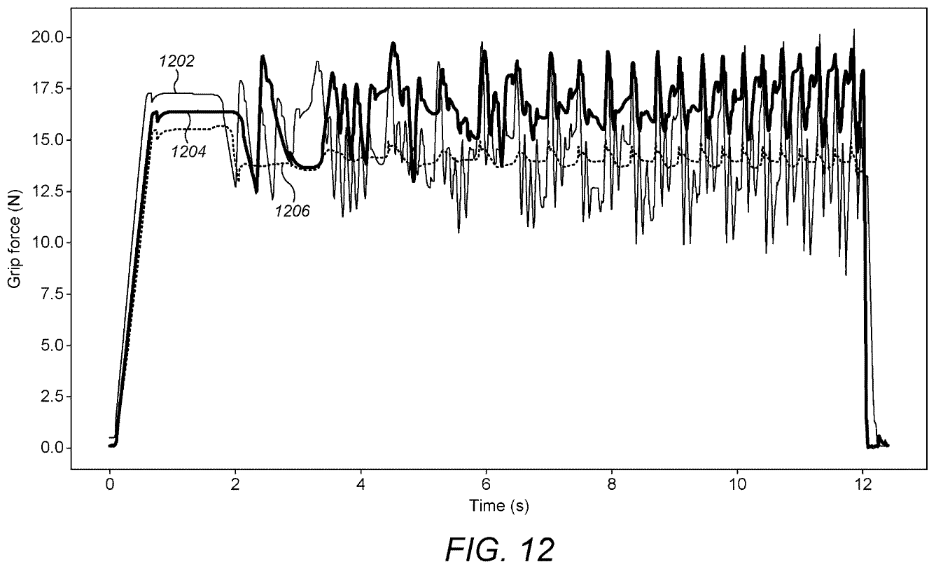

The benefits of the techniques discussed herein are illustrated in FIG. 12. FIG. 12 shows grip force changes for a conventional system in which the present control scheme is not implemented 1202, for a first system in which static control is implemented 1204, and for a second system in which dynamic control is implemented 1206. As can be seen, use of the present control scheme leads to improvements in the grip force applied, by reducing the grip force variations. Use of the dynamic control scheme offers further improvements compared to use of the static control scheme.

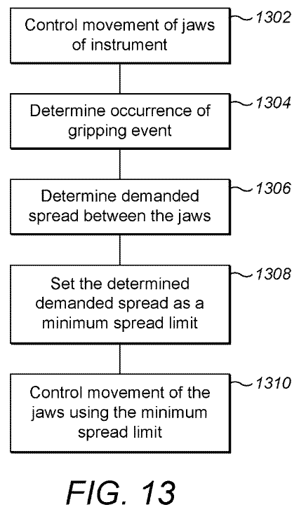

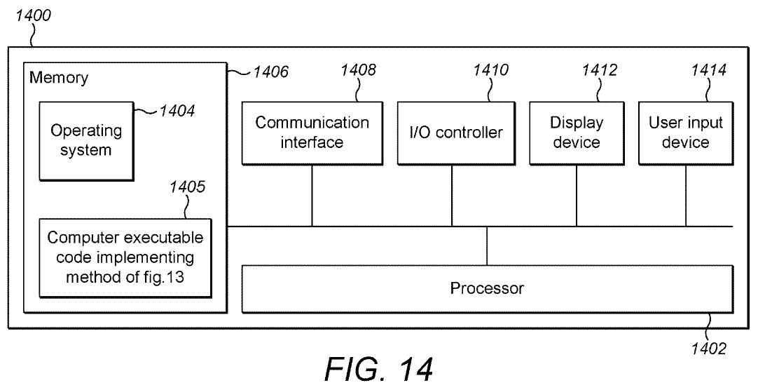

The method described herein will now be discussed with reference to FIG. 13. The method comprises controlling movement of the jaws according to a demanded spread between the jaws 1302. The method further comprises determining an occurrence of a gripping event as the jaws are driven relative to each other in the closing direction so as to apply a grip force between the jaws 1304. The method further comprises determining a demanded spread between the jaws on determining the occurrence of the gripping event 1306. The method also comprises setting the determined demanded spread as a minimum spread limit for the jaws 1308. The method comprises controlling movement of the jaws using the minimum spread limit so as to control grip force applied by the jaws 1310.

Instead of, or in addition to, determining the demanded spread between the jaws on determining the occurrence of the gripping event, the method may comprise determining a motor position, e.g. of a drive motor that controls movement of a jaw.

It is to be noted that the present techniques can be applied both to position control and to force control. In the context of a force controller, the method may comprise, e.g., controlling a force applied by the jaws of the instrument, determining the occurrence of a gripping event, determining a demanded and/or measured force between the jaws (and/or a tension in one or more driving elements), setting the determined force as a maximum demanded force limit, and controlling the force applied by the jaws using the determined maximum demanded force limit.

In some implementations, a combination of position control and force control can be used. An example method of such an implementation comprises controlling movement of the jaws 1302, determining the occurrence of a gripping event 1304, determining a demanded and/or measured force between the jaws (and/or a tension in one or more driving elements), setting the determined force as a maximum demanded force limit, and controlling movement of the jaws using the maximum demanded force limit so as to control grip force applied by the jaws.

Reference is now made to FIG. 14 which illustrates various components of an exemplary computing-based device 1400 which may be implemented as any form of a computing and/or electronic device, and in which embodiments of the methods and modification systems described herein may be implemented. The computing-based device 1400 comprises one or more processor 1402 which may be microprocessors, controllers or any other suitable type of processors for processing computer executable instructions. In some examples, for example where a system on a chip architecture is used, the processors 1402 may include one or more fixed function blocks (also referred to as accelerators) which implement a part of the method of modifying a data stream in hardware (rather than software or firmware). Platform software comprising an operating system 1404 or any other suitable platform software may be provided at the computing-based device to enable application software, such as software 1405 implementing the method of FIG. 13, to be executed on the device.

The computer executable instructions may be provided using any computer-readable media that is accessible by computing-based device 1400. Computer-readable media may include, for example, computer storage media such as memory 1406 and communications media. Computer storage media (i.e. non-transitory machine-readable media), such as memory 1406, includes volatile and non-volatile, removable and non-removable media implemented in any method or technology for storage of information such as computer readable instructions, data structures, program modules or other data. Computer storage media includes, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing-based device. In contrast, communication media may embody computer readable instructions, data structures, program modules, or other data in a modulated data signal, such as a carrier wave, or other transport mechanism. As defined herein, computer storage media does not include communication media. Although the computer storage media (i.e. non-transitory machine-readable media, e.g. memory 1406) is shown within the computing-based device 1400 it will be appreciated that the storage may be distributed or located remotely and accessed via a network or other communication link (e.g. using communication interface 1408).

The computing-based device 1400 also comprises an input/output controller 1410 arranged to output display information to a display device 1412 which may be separate from or integral to the computing-based device 1400. The display information may provide a graphical user interface. The input/output controller 1410 is also arranged to receive and process input from one or more devices, such as a user input device 1414 (e.g. a mouse or a keyboard). This user input may be used to initiate verification. In an embodiment the display device 1412 may also act as the user input device 1414 if it is a touch sensitive display device. The input/output controller 1410 may also output data to devices other than the display device, e.g. a locally connected printing device (not shown).

In the description above actions taken by the system have been split into functional blocks or modules for ease of explanation. In practice, two or more of these blocks could be architecturally combined. The functions could also be split into different functional blocks.

The present techniques have been described in the context of surgical robotic systems, though at least some features described are not limited to such systems, but may be applied to robotic systems more generally. In some examples, the present techniques may be applied to robotic systems that operate remotely. Some examples of situations in which the present techniques may be useful include those that make use of ‘snake-like’ robots for exploration, investigation or repair. In the case of a surgical robot the end effector could be a surgical tool such as a scissors, surgical cutter, surgical pincer or cauteriser.

Robotic systems can include manufacturing systems, such as vehicle manufacturing systems, parts handling systems, laboratory systems, and manipulators such as for hazardous materials or surgical manipulators.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A grip force controller for controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the grip force controller being configured to:
control movement of the jaws according to a demanded spread between the jaws;
determine an occurrence of a gripping event as the jaws are driven relative to each other in the closing direction so as to apply a grip force between the jaws;
determine a demanded spread between the jaws on determining the occurrence of the gripping event;
set the determined demanded spread as a minimum spread limit for the jaws; and
control movement of the jaws using the minimum spread limit so as to control grip force applied by the jaws.

2. The grip force controller according to claim 1, configured to determine the occurrence of the gripping event on determining that any one or more of the following occur:
a grip force between the jaws exceeds a grip force threshold;
a demanded motor force of a motor of the drive assembly for transferring drive to one of the jaws exceeds a motor force threshold;
an average demanded motor force of a plurality of motors of the drive assembly for transferring drive to respective ones of the jaws exceeds an average motor force threshold;
the motor of the drive assembly for transferring drive to one of the jaws approaches or reaches a saturation point;
a current through the motor of the drive assembly for transferring drive to one of the jaws exceeds a current threshold;
a driving element of the instrument, by which drive is transferred to one of the jaws, approaches or reaches a physical limit of the driving element; and
a physical limit is reached of a component of the instrument.

3. The grip force controller according to claim 2, configured to determine the occurrence of the gripping event based on a signal indicative of one or more of:
the grip force between the jaws;
the demanded motor force of the motor;
the average demanded motor force of the plurality of motors;
motor saturation of the motor of the drive assembly for transferring drive to one of the jaws;
current through the motor of the drive assembly for transferring drive to one of the jaws; and
tension in the driving element of the instrument by which drive is transferred to one of the jaws.

4. The grip force controller according to claim 3, in which:
the signal comprises or is derived from visual servoing based on an image of the instrument; and/or
the grip force controller is configured to filter the signal and to determine the occurrence of the gripping event based on the filtered signal.

5. The grip force controller according to claim 1, in which the minimum spread limit is a negative spread.

6. The grip force controller according to claim 1, configured to reset the minimum spread limit on determining that any one or more of the following occur:
the jaws are opened past an opening spread threshold;
the demanded spread exceeds a demanded spread threshold;
the demanded spread becomes positive;
a measured spread becomes positive;
the grip force applied between the jaws reduces past a further grip force threshold;
a tension of a driving element or other component of the instrument reduces past a reset threshold tension;
a torsion of a driving element or other component of the instrument reduces past a reset threshold torsion;
a flexing of a driving element or other component of the instrument reduces past a reset threshold flexing;
the jaws move relative to each other in the opening direction at a speed above an opening speed threshold;
a commanded jaw movement in the opening direction exceeds a threshold commanded jaw movement;
a change in force per unit of displacement exceeds a predetermined value;
a grip force commanded by the surgeon decreases;
a predetermined time, tᵣₑₛₑₜ, elapses; and
an indication of an end of a gripping operation is received.

7. The grip force controller according to claim 2, configured to determine that the instrument end effector is in a static condition where the average demanded motor force exceeds a static condition motor force.

8. The grip force controller according to claim 7, in which:
the determination that the instrument end effector is in a static condition is made on determining the occurrence of the gripping event; and/or
the static condition motor force is −180 units of effective force, and the average demanded motor force exceeds this when it becomes more negative than this value.

9. The grip force controller according to claim 1, configured to:
determine a yaw difference value based on a difference between a demanded yaw angle of the instrument end effector and a measured yaw angle of the instrument end effector; and
determine that the instrument end effector is in a dynamic condition where it is determined that the yaw difference value is greater than a dynamic yaw value.

10. The grip force controller according to claim 1, configured to determine that the instrument end effector is in a dynamic condition where it is determined that a yaw speed is greater than a dynamic yaw speed value.

11. The grip force controller according to claim 2, configured to determine that the instrument end effector is in a dynamic condition where the demanded motor force for one of the plurality of motors is greater than the motor force threshold and the demanded motor force for another of the plurality of motors is less than the motor force threshold.

12. The grip force controller according to claim 9, in which, where it is determined that the instrument end effector is in a dynamic condition, the minimum spread limit for the jaws is made more positive.

13. The grip force controller according to claim 12, in which the minimum spread limit is not increased past a spread value corresponding to a minimum grip force threshold, thereby retaining a minimum grip force between the jaws.

14. The grip force controller according to claim 9, in which a spread-dependent gain is applied to the demanded yaw angle.

15. The grip force controller according to claim 1, configured to:
determine a total force, F, on driving elements for driving each jaw;
determine a rate of change of the total force, F, with spread of the jaws, S;
determine a rate of change of spread of the jaws, S, with time, t;
in dependence on the determined rate of change of the total force exceeding a predetermined constant, k_{S}, and the determined rate of change of spread of the jaws exceeding another predetermined constant, V_{S}, setting the instantaneous total force as a baseline force and setting the instantaneous spread as a baseline spread.

16. The grip force controller according to claim 15, configured to determine a change in total force relative to the baseline force, and to determine the occurrence of the gripping event where the determined change in total force exceeds a threshold force value.

17. The grip force controller according to claim 15, configured to reset the baseline force where it is determined that the spread of the jaws increases past the baseline spread and/or increases to a positive spread.

18. A method of controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the method comprising:
controlling movement of the jaws according to a demanded spread between the jaws;
determining an occurrence of a gripping event as the jaws are driven relative to each other in the closing direction so as to apply a grip force between the jaws;
determining a demanded spread between the jaws on determining the occurrence of the gripping event;
setting the determined demanded spread as a minimum spread limit for the jaws; and
controlling movement of the jaws using the minimum spread limit so as to control grip force applied by the jaws.

19. A grip force controller for controlling grip force of an instrument in a surgical robotic system, the surgical robotic system comprising a robot having a base and an arm extending from the base to a drive assembly for engaging with the instrument, the arm comprising a plurality of joints whereby the configuration of the arm can be altered, the instrument having an end effector comprising a plurality of jaws and the drive assembly being for transferring drive to the plurality of jaws of the instrument to drive the jaws relative to each other in a closing direction for gripping an object between the jaws and in an opening direction for releasing a gripped object, the grip force controller being configured to:
control a force applied between the jaws according to a demanded force;
determine an occurrence of a gripping event as the jaws are driven relative to each other in the closing direction so as to apply a grip force between the jaws;
determine an indication of a demanded force between the jaws on determining the occurrence of the gripping event;
set the determined demanded force as a maximum demanded force limit for the jaws; and
control the force applied between the jaws using the maximum demanded force limit so as to control grip force applied by the jaws.

20. (canceled)
